(19) United States
(12) Patent Application Publication
(10) Pub. No.: US 2009/0169644 A1
(43) Pub. Date: Jul. 2, 2009

(54) Antidandruff Shampoo

(75) Inventors: Dieter Goddinger, Klein Nordende (DE); Thomas Schroeder, Hamburg (DE)

Correspondence Address: Ratner Prestia, P.O. Box 980, Valley Forge, PA, 19482, US

(73) Assignee: Henkel AG & Co. KGaA, Duesseldorf (DE)

(21) Appl. No.: 12/351,306

(22) Filed: Jan. 9, 2009

Related U.S. Application Data
(63) Continuation of application No. PCT/EP2007/002868, filed on Mar. 30, 2007.

(30) Foreign Application Priority Data
Jul. 12, 2006 (DE) 10 2006 032 505.2

(51) Int. Cl. A61K 33/30; A61K 47/06; A61K 31/496; A61K 31/555; A61K 31/60; A61K 31/44; A61P 31/10

(52) U.S. Cl. 424/642; 514/785; 514/254.07; 514/186; 514/159; 514/345

(57) ABSTRACT

Surfactant-containing cleansing compositions contain hydrogenated castor oil for stabilizing an antidandruff component that is insoluble in the cleansing composition. As a result, thorough and gentle cleansing of skin and/or of hair with simultaneous antidandruff effect is ensured, without phase separation, change in viscosity, or reduction in the foaming ability during storage. Optimally, it is possible to dispense with additional stabilization of the antidandruff component through the use of gel formers.

### CROSS REFERENCE TO RELATED APPLICATIONS

This application is a continuation under 35 U.S.C. §§ 120 and 365(c) of International Application PCT/EP2007/002868, filed on Mar. 30, 2007. This application also claims priority under 35 U.S.C. § 119 of DE 10 2006 032 505.2 filed on Jul. 12, 2006. The disclosures of PCT/EP2007/002868 and DE 10 2006 032 505.2 are incorporated by reference in their entirety.

### BACKGROUND OF THE INVENTION

The invention relates to surfactant-containing cleansing agents, which comprise hardened castor oil for stabilizing an antidandruff agent which is insoluble therein, and to a method of stabilizing insoluble antidandruff agents in surfactant-containing formulations.

Cleansing agents for skin and hair, as are commercially available for example in the form of liquid soaps, shampoos, shower foams, bath foams, shower and washing gels, have not only to display a good cleansing capacity but should furthermore display good compatibility with the skin and the hair and not lead to severe degreasing or dryness even when used frequently.

Conventionally, skin and hair cleansing agents are formulated on the basis of anionic, nonionic and/or amphoteric interfacially active substances, but very particularly preferably on the basis of anionic surfactants. Although such compositions display good washing power, the cosmetic properties associated therewith are inadequate, in particular due to the removal of skin- and hair-intrinsic lipids and proteins from the surface of the skin or hair.

Dandruff control is a further important aspect of hair cleansing and care. In general, antidandruff treatment is understood to mean the control of Pityrosporum ovale yeast fungus, which causes cosmetic dandruff when it occurs in excess. The solid zinc pyrithione has proven to be an effective antidandruff agent, but this has to be stabilized in the shampoo matrix, since it is also present in solid, undissolved form in the shampoo matrix. Without sufficient stabilization, the active ingredient would otherwise settle out and the shampoo would thus lose its antidandruff action.

In the literature and in practice it is known to stabilize zinc pyrithione effectively by using acrylate polymers (Carbopol types) or xanthan gum gel formers.

However, the use of gel formers has disadvantages, since these have a negative effect on the foaming of a surfactant-containing cleansing agent.

### DESCRIPTION OF THE INVENTION

It was therefore an object of the present invention to produce a surfactant-containing cleansing agent for skin and hair which not only ensures thorough, gentle cleansing of the skin/scalp and/or the hair but at the same time has an antidandruff action and is distinguished with regard to application by stable dispersion of the antidandruff agent in the shampoo matrix.

Ideally, in the light of the above-stated disadvantages, it is intended to be able to dispense with additional stabilization of the antidandruff agent by gel formers.

This object was achieved by incorporating hardened castor oil into the surfactant-containing cleansing agent for stabilizing the insoluble antidandruff agent.

According to the invention, cleansing agents are preferred which comprise a mixture of at least one anionic surfactant with an amphoteric and/or zwitterionic and/or nonionic surfactant.

The total surfactant content in the cleansing agent amounts 5 to 35%, preferably 7 to 25% and in particular 8 to 15%, relative to the total weight of the cleansing agent.

Anionic surfactants which are suitable in preparations according to the invention are any anionic surface-active substances suitable for use on the human body. These are characterized by an anionic water-solubilizing group such as for example a carboxylate, sulfate, sulfonate or phosphate group and a lipophilic alkyl group having some 8 to 30 C atoms. The molecule may additionally contain glycol or polyglycol ether groups, ester, ether and amide groups and hydroxyl groups. Examples of suitable anionic surfactants are, in each case in the form of sodium, potassium and ammonium and the mono-, di- and trialkanolammonium salts having 2 to 4 C atoms in the alkanol group,
linear and branched fatty acids with 8 to 30 C atoms (soaps),
ether carboxylic acids of the formula R—O—(CH₂—CH₂O)ₓ—CH₂—COOH, in which R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfofatty acid methyl esters of fatty acids having 8 to 30 C atoms,
alkyl sulfates and alkyl polyglycol ether sulfates of the formula R—O(CH₂—CH₂O)ₓ—OSO₃H, in which R is a preferably linear alkyl group having 8 to 30 C atoms and x=0 or 1 to 12,
mixtures of surface-active hydroxysulfonates according to DE-A-37 25 030,
sulfated hydroxyalkyl polyethylene and/or hydroxyalkylene propylene glycol ethers according to DE-A-37 23 354,
sulfonates of unsaturated fatty acids having 8 to 24 C atoms and 1 to 6 double bonds according to DE-A-39 26 344,
esters of tartaric acid and citric acid with alcohols, which are addition products of approx. 2-15 molecules of ethylene oxide and/or propylene oxide onto fatty alcohols having 8 to 22 C atoms,
alkyl- and/or alkenyl ether phosphates of the formula (II),

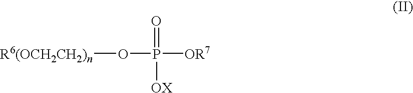

in which R⁶ preferably denotes an aliphatic hydrocarbon residue with 8 to 30 carbon atoms, R⁷ denotes hydrogen, a residue (CH₂CH₂O)ₙR⁶ or X, n denotes numbers from 1 to 10 and X denotes hydrogen, an alkali or alkaline earth metal or NR⁸R⁹R¹⁰R¹¹, with R⁸ to R¹¹ mutually independently denoting hydrogen or a C₁ to C₄ hydrocarbon residue, sulfated fatty acid alkylene glycol esters of the formula (III),

R¹²CO(AlkO)ₙSO₃M (III)
in which R¹²CO— denotes a linear or branched, aliphatic, saturated and/or unsaturated acyl residue with 6 to 22 C atoms, Alk denotes CH₂CH₂, CHCH₃CH₂ and/or CH₂CHCH₃, n denotes numbers from 0.5 to 5 and M denotes a cation, as they are described in DE-OS 197 36 906.5,
monoglyceride sulfates and monoglyceride ether sulfates of the formula (IV), as have for example been described in EP-B1 0 561 825, EP-B1 0 561 999, DE-A1 42 04 700 or by A. K. Biswas et al. in J. Am. Oil Chem. Soc. 37, 171 (1960) and F. U. Ahmed in J. Am. Oil Chem. Soc. 67, 8 (1990),

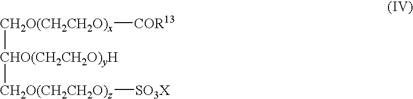

in which R¹³CO denotes a linear or branched acyl residue having 6 to 22 carbon atoms, x, y and z in total denote 0 or denote numbers from 1 to 30, preferably 2 to 10, and X denotes an alkali or alkaline earth metal. Typical examples of monoglyceride (ether) sulfates suitable for the purposes of the invention are the reaction products of lauric acid monoglyceride, coconut fatty acid monoglyceride, palmitic acid monoglyceride, stearic acid monoglyceride, oleic acid monoglyceride and tallow fatty acid monoglyceride and the ethylene oxide addition products thereof with sulfur trioxide or chlorosulfonic acid in the form of the sodium salts thereof. Preferably, monoglyceride sulfates of the formula (VIII) are used, in which R¹³CO for a linear acyl residue with 8 to 18 carbon atoms.

Preferred anionic surfactants are alkyl sulfates, alkyl polyglycol ether sulfates and ether carboxylic acids having 10 to 18 C atoms in the alkyl group and up to 12 glycol ether groups per molecule, sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups.

Particularly preferred anionic surfactants are the alkali metal or ammonium salts of lauryl ether sulfate with a degree of ethoxylation of 2 to 4 EO.

Those surface-active compounds which bear at least one quaternary ammonium group and at least one —COO⁽⁻⁾ or —SO₃⁽⁻⁾ group on each molecule are designated as zwitterionic surfactants. Particularly suitable zwitterionic surfactants are “betaines” such as N-alkyl N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines having in each case 8 to 18 C atoms in the alkyl or acyl group and cocoacylaminoethylhydroxyethylcarboxymethyl glycinate. One preferred zwitterionic surfactant is the fatty acid amide derivative known by the INCI name Cocamidopropyl Betaine.

Amphoteric surfactants are taken to mean those surface-active compounds which, in addition to a C₈-C₂₄ alkyl or acyl group, contain at least one free amino group and at least one —COOH or —SO₃H group per molecule and are capable of forming internal salts. Examples of suitable ampholytic surfactants are N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids having in each case approx. 8 to 24 C atoms in the alkyl group. Particularly preferred ampholytic surfactants are N-cocoalkyl aminopropionate, cocoacylaminoethyl aminopropionate and C₁₂₋₁₈-acyl sarcosine.

Nonionic surfactants contain as hydrophilic group for example a polyol group, a polyalkylene glycol ether group or a combination of a polyol group and polyglycol ether group. Such compounds are for example
addition products of 2 to 50 mol of ethylene oxide and/or 0 to 5 mol of propylene oxide onto linear and branched fatty alcohols having 8 to 30 C atoms, onto fatty acids having 8 to 30 C atoms and onto alkylphenols having 8 to 15 C atoms in the alkyl group,
addition products, end group-terminated with a methyl or C₂-C₆ alkyl residue, of 2 to 50 mol of ethylene oxide and/or 0 to 5 mol of propylene oxide onto linear and branched fatty alcohols having 8 to 30 C atoms, onto fatty acids having 8 to 30 C atoms and onto alkylphenols having 8 to 15 C atoms in the alkyl group, such as for example the grades obtainable under the commercial names Dehydrol® LS, Dehydrol® LT (Cognis),
C₁₂-C₃₀ fatty acid mono- and diesters of addition products of 1 to 30 mol of ethylene oxide onto glycerol,
addition products of 5 to 60 mol of ethylene oxide onto castor oil and hardened castor oil, for example hydrogenated castor oil+40 EO, as is commercially available for example under the trade name Cremophor CO 455 from SHC,
polyol fatty acid esters, such as for example the commercial product Hydagen® HSP (Cognis) or Sovermol grades (Cognis),
alkoxylated triglycerides,
alkoxylated fatty acid alkyl esters of the formula (V)

R¹⁴CO—(OCH₂CHR¹⁵)_{w}OR¹⁶ (V)
in which R¹⁴CO denotes a linear or branched, saturated and/or unsaturated acyl residue having 6 to 22 carbon atoms, R¹⁵ denotes hydrogen or methyl, R¹⁶ denotes linear or branched alkyl residues having 1 to 4 carbon atoms and w denotes numbers from 1 to 20,
amine oxides,
hydroxy mixed ethers, as are for example described in DE-OS 19738866,
sorbitan fatty acid esters and addition products of ethylene oxide onto sorbitan fatty acid esters such as for example polysorbates,
sugar fatty acid esters and addition products of ethylene oxide onto sugar fatty acid esters,
addition products of ethylene oxide onto fatty acid alkanolamides and fatty amines,
fatty acid N-alkylglucamides,
alkylpolyglycosides corresponding to the general formula RO-(Z)ₓ wherein R denotes alkyl, Z denotes sugar and x denotes the number of sugar units. Alkylpolyglycosides usable according to the invention may for example contain only one specific alkyl residue R. Conventionally, however, these compounds are produced starting from natural fats and oils or mineral oils. In this case, alkyl residues R which are present are mixtures corresponding to the starting compounds or corresponding to the particular processing of these compounds.

Particularly preferred such alkylpolyglycosides are those in which R

substantially consists of C₈ and C₁₀ alkyl groups,

substantially consists of C₁₂ and C₁₄ alkyl groups,

substantially consists of C₈ to C₁₆ alkyl groups or

substantially consists of C₁₂ to C₁₆ alkyl groups or

substantially consists of C₁₆ to C₁₈ alkyl groups.

The sugar building block Z used may be any desired mono- or oligosaccharides. Sugars having 5 or 6 carbon atoms and the corresponding oligosaccharides are conventionally used. Such sugars are for example glucose, fructose, galactose, arabinose, ribose, xylose, lyxose, allose, altrose, mannose, gulose, idose, talose and sucrose. Preferred sugar building blocks are glucose, fructose, galactose, arabinose and sucrose; glucose is particularly preferred.

Alkylpolyglycosides usable according to the invention contain on average 1.1 to 5 sugar units. Alkylpolyglycosides with x values of 1.1 to 2.0 are preferred. Alkylglycosides in which x is 1.1 to 1.8 are very particularly preferred.

The alkoxylated homologues of the stated alkylpolyglycosides may also be used according to the invention. These homologues may contain on average up to 10 ethylene oxide and/or propylene oxide units per alkylglycoside unit.

Preferred nonionic surfactants have proved to be alkylene oxide addition products onto saturated linear fatty alcohols and fatty acids with in each case 2 to 30 mol of ethylene oxide per mol of fatty alcohol or fatty acid respectively. Preparations having excellent properties are likewise obtained if they contain fatty acid esters of ethoxylated glycerol as the nonionic surfactants.

These compounds are characterized by the following parameters. The alkyl residue R contains 6 to 22 carbon atoms and may be both linear and branched. Primary linear aliphatic residues and those methyl-branched in position 2 are preferred. Such alkyl residues are for example 1-octyl, 1-decyl, 1-lauryl, 1-myristyl, 1-cetyl and 1-stearyl. 1-Octyl, 1-decyl, 1-lauryl, 1-myristyl are particularly preferred. When “oxo alcohols” are used as starting materials, compounds having an uneven number of carbon atoms in the alkyl chain predominate.

The compounds with alkyl groups used as surfactant may in each case comprise uniform substances. It is, however, generally preferred to start from native plant or animal raw materials when producing these substances, such that mixtures of substances having a differing alkyl chain length depending on the particular raw material are obtained.

The surfactants, which are addition products of ethylene and/or propylene oxide onto fatty alcohols or derivatives of these addition products, may be used both as products with a “normal” homologue distribution and as products with a narrow homologue distribution. A “normal” homologue distribution is here taken to mean mixtures of homologues which are obtained on reacting fatty alcohol and alkylene oxide using alkali metals, alkali metal hydroxides or alkali metal alkoxides as catalysts. Narrow homologue distributions, in contrast, are obtained if hydrotalcite, alkaline earth metal salts of ether carboxylic acids, alkaline earth metal oxides, hydroxides or alkoxides are for example used as catalysts. It may be preferred to use products with a narrow homologue distribution.

According to a further embodiment of the invention, the surfactant-containing cleansing agents additionally contain cationic surfactants of the type comprising quaternary ammonium compounds, ester quats and amidoamines.

Preferred quaternary ammonium compounds are ammonium halides, in particular chlorides and bromides, such as alkyltrimethylammonium chlorides, dialkyldimethylammonium chlorides and trialkylmethylammonium chlorides, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, and the imidazolium compounds known by the INCI names quaternium-27 and quaternium-83. The long alkyl chains of the above-stated surfactants preferably comprise 10 to 18 carbon atoms.

Ester quats are known substances which contain both at least one ester function and at least one quaternary ammonium group as a structural element. Preferred ester quats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold, for example, under the trademarks Stepantex®, Dehyquart® and Armocare®. The products Armocare® VGH-70, an N,N-bis(2-palmitoyloxyethyl)dimethylammonium chloride, and Dehyquart® F-75, Dehyquart® C-4046, Dehyquart® L80 and Dehyquart® AU-35 are examples of such ester quats.

The alkylamidoamines are conventionally produced by amidating natural or synthetic fatty acids and fatty acid cuts with dialkylaminoamines. One compound from this group of substances which is particularly suitable according to the invention is stearamidopropyldimethylamine which is commercially available under the name Tegoamid® S 18.

The cationic surfactants are contained in the agents according to the invention preferably in quantities of from 0.05 to 10 wt. %, relative to the total agent. Quantities of 0.1 to 5 wt. % are particularly preferred.

The antidandruff agent in the surfactant-containing cleansing agents is preferably selected from ketoconazole, climbazole, zinc pyridinethione, salicylic acid, zinc carbonates and piroctone olamine.

It is used in the agents according to the invention, relative to the total weight, in a quantity of from 0.05 to 2.0%, preferably in a quantity of from 0.25 to 1.5% and in particular in a quantity of from 0.5 to 1.0%.

In a particularly preferred embodiment of the invention the antidandruff agent is zinc pyrithione.

Preferably the zinc pyrithione has a particle size of from 0.1 to 10 μm, preferably from 0.5 to 5 μm, and in particular from 0.8 to 3 μm.

In addition to the antidandruff agent and the surfactants, hardened castor oil is a third absolutely essential component in the cleansing agents according to the invention.

According to the invention, this is preferably the product known under the INCI name of “Hydrogenated Castor Oil”, as is commercially available for example from Cognis under the name Cutina HR®.

The hydrogenated castor oil is used in the compositions according to the invention, relative to the total weight thereof, in a quantity of from 0.01 to 5%, preferably in a quantity of from 0.05 to 3% and in particular in a quantity of from 0.1 to 2%.

In a particularly preferred embodiment of the invention, the surfactant-containing cleansing agents additionally contain at least one cationic polymer.

Cationic polymers should be taken to mean polymers which comprise groups in the main and/or side chain which may be “temporarily” or “permanently” cationic. Polymers which are designated “permanently cationic” according to the invention are those which, irrespective of the pH value of the agent, comprise a cationic group. As a rule, these are polymers which contain a quaternary nitrogen atom, for example in the form of an ammonium group. Preferred cationic groups are quaternary ammonium groups. Polymers which have proven particularly suitable are those in which the quaternary ammonium group is bound via a C₁₋₄ hydrocarbon group to a main polymer chain synthesized from acrylic acid, methacrylic acid or the derivatives thereof.

Homopolymers of the general formula (IV),

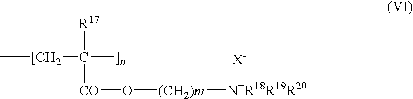

in which R¹⁷ is —H or —CH₃, R¹⁸, R¹⁹ and R²⁰ are mutually independently selected from C₁₋₄ alkyl, alkenyl or hydroxyalkyl groups, m=1, 2, 3 or 4, n is a natural number and X⁻ a physiologically acceptable organic or inorganic anion, and copolymers substantially consisting of the monomer units listed in formula (III) and nonionogenic monomer units are particularly preferred cationic polymers. In the context of these polymers, those which are preferred according to the invention are those for which at least one of the following conditions applies:

R¹⁷ denotes a methyl group

R¹⁸, R¹⁹ and R²⁰ denote methyl groups

m has the value 2.

Physiologically acceptable counterions X⁻ which may, for example, be considered are halide ions, sulfate ions, phosphate ions, methosulfate ions and organic ions such as lactate, citrate, tartrate and acetate ions. Halide ions, in particular chloride, are preferred.

A suitable homopolymer is poly(methacryloyloxyethyltrimethylammonium chloride), which may if desired be crosslinked, with the INCI name of polyquarternium-37. Crosslinking may if desired proceed with the assistance of olefinically polyunsaturated compounds, for example divinylbenzene, tetraallyloxyethane, methylenebisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylenebisacrylamide is a preferred crosslinking agent.

The homopolymer is preferably used in the form of a nonaqueous polymer dispersion which should have a polymer fraction of no less than 30 wt. %. Such polymer dispersions are commercially available under the names Salcare® SC 95 (approx. 50% polymer fraction, further components: mineral oil (INCI name: Mineral Oil) and tridecyl-polyoxypropylenepolyoxyethylene ether (INCI name: PPG-1-Trideceth-6)) and Salcare® SC 96 (approx. 50% polymer fraction, further components: mixture of diesters of propylene glycol with a mixture of caprylic and capric acid (INCI name: Propylene Glycol Dicaprylate/Dicaprate) and tridecyl-polyoxypropylenepolyoxyethylene ether (INCI name: PPG-1-Trideceth-6)).

Copolymers with monomer units according to the formula (VI) preferably contain acrylamide, methacrylamide, acrylic acid C₁₋₄ alkyl esters and methacrylic acid C₁₋₄ alkyl esters as nonionogenic monomer units. Acrylamide is particularly preferred among these nonionogenic monomers. These copolymers, as described above for the homopolymers, may also be crosslinked. A copolymer which is preferred according to the invention is crosslinked acrylamide-methacryloyloxyethyltrimethylammonium chloride copolymer. Such copolymers, in which the monomers are present in a weight ratio of approx 20:80, are commercially available as approx. 50% nonaqueous polymer dispersions under the name Salcare® SC 92.

Further preferred cationic polymers are for example
quaternized cellulose derivatives, as are commercially available under the names Celquat® and Polymer JR®. The compounds Celquat® H 100, Celquat® L 200 and polymer JR®400 are preferred quaternized cellulose derivatives,
cationic alkyl polyglycosides according to DE-PS 44 13 686,
cationized honey, for example the commercial product Honeyquat® 50, cationic guar derivatives, such as in particular the products distributed under the trade names Cosmedia®Guar and Jaguar®,
polysiloxanes with quaternary groups, such as for example the commercially obtainable under the names Q2-7224 (manufacturer: Dow Corning; a stabilized trimethylsilylamodimethicone), Dow Corning® 929 Emulsion (containing a hydroxylamine-modified silicone which is also designated an amodimethicone), SM-2059 (manufacturer: General Electric), SLM-55067 (manufacturer: Wacker) and Abil®-Quat 3270 and 3272 (manufacturer: Th. Goldschmidt; diquaternary polydimethylsiloxane, Quaternium-80).
polymeric dimethyldiallylammonium salts and the copolymers thereof with esters and amides of acrylic acid and methacrylic acid. The products commercially available under the names Merquat®100 (poly(dimethyldiallylammonium chloride)) and Merquat®550 (dimethyldiallylammonium chloride-acrylamide copolymer) are examples of such cationic polymers,
copolymers of vinylpyrrolidone with quaternized derivatives of dialkylaminoalkyl acrylate and methacrylate, such as for example vinyl pyrrolidone-dimethylaminoethyl methacrylate copolymers quaternized with diethyl sulfate. Such compounds are commercially available under the names Gafquat®734 and Gafquat®755,
vinylpyrrolidone-vinylimidazolium methochloride copolymers, as are offered for sale under the names Luviquat® FC 370, FC 550, FC 905 and HM 552,
quaternized polyvinyl alcohol, and the polymers known under the names
Polyquarternium 2,
Polyquarternium 17,
Polyquarternium 18 and
Polyquarternium 27 with quaternary nitrogen atoms in the polymer main chain.

The polymers known under the names Polyquarternium-24 (commercial product, for example Quatrisoft® LM 200) may also be used as cationic polymers. Copolymers of vinylpyrrolidone, as are available as commercial products Copolymer 845 (manufacturer: ISP), Gaffix® VC 713 (manufacturer: ISP), Gafquat®ASCP 1011, Gafquat®HS 110, Luviquat®8155 and Luviquat® MS 370 may likewise be used according to the invention.

Further cationic polymers according to the invention are “temporarily cationic” polymers. These polymers conventionally contain an amino group which at specific pH values assumes the form of a quaternary ammonium group and is thus cationic. Chitosan and the derivatives thereof are for example preferred, as are readily commercially available for example under the trade names Hydagen® CMF, Hydagen® HCMF, Kytamer® PC and Chitolam® NB/101. Chitosans are deacetylated chitins which are commercially available in various degrees of deacetylation and various degrees of degradation (molecular weights). The production thereof is described, for example, in DE 44 40 625 A1 and in DE 1 95 03 465 A1

Particularly highly suitable chitosans exhibit a degree of deacetylation of at least 80% and a molecular weight of 5·10⁵ to 5·10⁶ (g/mol).

In order to produce preparations according to the invention, the chitosan must be converted into the salt form. This may proceed by dissolution in dilute aqueous acids. Suitable acids are both mineral acids such as for example hydrochloric acid, sulfuric acid and phosphoric acid and organic acids, for example low molecular weight carboxylic acids, polycarboxylic acids and hydroxycarboxylic acids. Relatively high molecular weight alkylsulfonic acids or alkylsulfuric acids or organophosphoric acids may furthermore be used, provided that they have the necessary physiological acceptability. Suitable acids for converting the chitosans into the salt form are for example acetic acid, glycolic acid, tartaric acid, malic acid, citric acid, lactic acid, 2-pyrrolidinone-5-carboxylic acid, benzoic acid or salicylic acid. Low molecular weight hydroxycarboxylic acids such as for example glycolic acid or lactic acid are preferably used.

In a particularly preferred embodiment of the invention, at least one polymer from the group Polyquarternium-7 (Merquat 550), Polyquarternium-6 or Polyquarternium-10 is contained in the cleansing agents as a cationic polymer.

The cationic polymers are contained in the agents according to the invention preferably in quantities of from 0.1 to 5 wt. %, relative to the total agent. Quantities of 0.2 to 3, in particular of 0.5 to 2 wt. %, are particularly preferred.

In a further preferred embodiment of the invention, the surfactant-containing cleansing agents additionally contain at least one constituent from the group of water-insoluble oil components, vitamins, provitamins, protein hydrolysates, plant extracts, UV filters, amino acids, water-insoluble silicones, water-soluble silicones and/or amodimethicones.

Suitable water-insoluble oil components according to the invention are vegetable, mineral or synthetic oils, and mixtures of these components.

Conventionally, triglycerides and mixtures of triglycerides are used as the natural (vegetable) oils. Preferred natural oils for the purposes of the invention are coconut oil, (sweet) almond oil, walnut oil, peach stone oil, avocado oil, tea tree oil, soy oil, sesame oil, sunflower oil, tsubaki oil, evening primrose oil, rice bran oil, palm kernel oil, mango kernel oil, lady's smock oil, thistle oil, macadamia nut oil, grapeseed oil, apricot kernel oil, babassu oil, olive oil, wheat germ oil, pumpkin seed oil, mallow oil, hazelnut oil, safflower oil, canola oil, sasanqua oil, jojoba oil and shea butter.

Mineral oils which are used are in particular mineral oils, paraffin and isoparaffin oils and synthetic hydrocarbons. One hydrocarbon usable according to the invention is for example the commercially available product 1,3-di-(2-ethyl hexyl)cyclohexane (Cetiol® S).

Synthetic oils which may be considered are silicone compounds, in particular dialkyl and alkylaryl silicones, such as for example dimethylpolysiloxane and methylphenylpolysiloxane, and the hydroxyterminated, alkoxylated and quaternized analogues thereof. Examples of such silicones are the products distributed by Dow Corning under the names DC 190, DC 200, DC 344 and DC 345 (Cyclomethicone).

A dialkyl ether may additionally serve as the oil component.

Dialkyl ethers which may be used according to the invention are in particular di-n-alkyl ethers having a total of between 12 to 36 C atoms, in particular 12 to 24 C atoms, such as for example di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and di-tert.-butyl ether, diisopentyl ether, di-3-ethyldecyl ether, tert.-butyl-n-octyl ether, iso-pentyl-n-octyl ether and 2-methylpentyl-n-octyl ether.

Particular preference is given according to the invention to di-n-octyl ether, which is commercially obtainable under the name Cetiol® OE.

The cleansing agents according to the invention contain the water-insoluble oil component preferably in a quantity range of from 0.1 to 5 wt. %, in particular from 0.5 to 2 wt. %, relative to the total weight of the agent.

In a further preferred embodiment of the invention, the action of the active ingredient combination according to the invention may be still further optimized by further fatty substances. Further fatty substances should be taken to mean fatty acids, fatty alcohols and natural and synthetic waxes, which may assume both solid form and liquid form in an aqueous dispersion.

Fatty acids which may be used are linear and/or branched, saturated and/or unsaturated fatty acids having 6-30 carbon atoms. Fatty acids having 10-22 carbon atoms are preferred. Such substances which may, for example, be mentioned are isostearic acid, such as the commercial products Emersol® 871 and Emersol® 875, and isopalmitic acids such as the commercial product Edenor® IP 95, and any further fatty acids distributed under the trade name Edenor® (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof, which arise for example during pressure splitting of natural fats and oils, during the oxidation of aldehydes from Roelen's oxo synthesis or the dimerization of unsaturated fatty acids. The fatty acid cuts obtainable from coconut oil or palm oil are conventionally particularly preferred; in general, it is particularly preferred to use stearic acid.

The quantity used here amounts to 0.1-15 wt. %, relative to the total agent. In a preferred embodiment, the quantity amounts to 0.5-10 wt. %, with quantities of 1-5 wt. % being very particularly advantageous.

Fatty alcohols which may be used are saturated, mono- or polyunsaturated, branched or unbranched fatty alcohols having C₆-C₃₀, preferably C₁₀-C₂₂ and very particularly preferably C₁₂-C₂₂ carbon atoms. Examples which may be used for the purposes of the invention are decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, caprylic alcohol, capric alcohol, linoleyl alcohol, linolenyl alcohol and behenyl alcohol, and the Guerbet alcohols thereof, this list being intended to be exemplary and not limiting in nature. The fatty alcohols are, however, preferably derived from natural fatty acids, it conventionally being possible to start by isolation from the fatty acid esters by reduction. It is also possible to use according to the invention those fatty alcohol cuts which are produced by reducing naturally arising triglycerides such as beef fat, palm oil, peanut oil, rapeseed oil, cottonseed oil, soy oil, sunflower oil and linseed oil or of fatty acids arising from the transesterification products thereof with corresponding alcohols, and thus constitute a mixture of different fatty alcohols. Such substances are commercially obtainable for example under the names Stenol®, for example Stenol® 1618 or Lanette® 0, or Lorol®, z.B. Lorol® C8, Lorol® C14, Lorol® C18, Lorol® C8-18, HD-Ocenol®, Crodacol®, z.B. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols, as are for example commercially obtainable under the names Corona®, White Swan®, Coronet® or Fluilan®), may, of course also be used according to the invention.

The fatty alcohols are used in quantities of 0.1-20 wt. %, relative to the entire preparation, preferably in quantities of 0.1-10 wt. %.

Natural or synthetic waxes which may be used according to the invention are solid paraffins or isoparaffins, carnauba waxes, beeswaxes, candelilla waxes, ozokerites, ceresin, spermaceti, sunflower wax, fruit waxes such as for example apple wax or citrus wax, or PE or PP microwaxes. Such waxes are obtainable for example through Kahl & Co., Trittau.

Further fatty substances are for example
ester oils. Ester oils should be taken to mean the esters of C₆-C₃₀ fatty acids with C₂-C₃₀ fatty alcohols. The monoesters of fatty acids with alcohols having 2 to 24 C atoms are preferred. Examples of fatty acid moieties used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and the technical mixtures thereof, which arise for example during pressure splitting of natural fats and oils, during the oxidation of aldehydes from Roelen's oxo synthesis or the dimerization of unsaturated fatty acids. Examples of the fatty alcohol moieties in the ester oils are isopropyl alcohol, caproic alcohol, caprylic alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof, which arise for example during the high pressure hydrogenation of industrial methyl esters based on fats and oils or aldehydes from Roelen's oxo synthesis and as a monomer fraction during the dimerization of unsaturated fatty alcohols. Particular preference is given according to the invention to isopropyl myristate (Rilanit® IPM), isononanoic acid C₁₆₋₁₈-alkyl ester (Cetiol® SN), 2-ethylhexyl palmitate, (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and diol esters such as ethylene glycol dioleate, ethylene glycol diisotridecanoate, propylene glycol di(2-ethyl hexanoate), propylene glycol diisostearate,
propylene glycol dipelargonate, butanediol diisostearate, neopentyl glycol dicaprylate, symmetrical, asymmetrical or cyclic esters of carbonic acid with fatty alcohols, for example described in DE-OS 197 56 454, glycerol carbonate or dicaprylyl carbonate (Cetiol® CC),
ethoxylated or unethoxylated mono-, di- and trifatty acid esters of saturated and/or unsaturated linear and/or branched fatty acids with glycerol, such as for example Monomuls® 90-018, Monomuls® 90-L12 or Cutina® MD.

The quantity used amounts to 0.1-50 wt. %, relative to the total agent, preferably 0.1-20 wt. % and particularly preferably 0.1-15 wt. %, relative to the total agent.

The total quantity of oil and fat components in the agents according to the invention conventionally amounts to 6-45 wt. %, relative to the total agent. Quantities of 10-35 wt. % are preferred according to the invention.

Vitamins, provitamins and vitamin precursors and the derivatives thereof preferred according to the invention are those representatives which are conventionally assigned to the groups A, B, C, E, F and H.

The group of substances designated vitamin A includes retinol (vitamin A₁) and 3,4-didehydroretinol (vitamin A₂). β-Carotene is the provitamin of retinol. Examples of substances which may be considered according the invention as the vitamin A component are vitamin A acid and the esters thereof, vitamin A aldehyde and vitamin A alcohol and the esters thereof such as the palmitate and the acetate. The agents according to the invention preferably contain the vitamin A component in quantities of from 0.01-1 wt. %, relative to the entire preparation.

The vitamin B group or the vitamin B complex includes, inter alia Vitamin B₁ (thiamin) Vitamin B₂ (riboflavine) Vitamin B₃. This designation is frequently used for the compounds nicotinic acid and nicotinamide (niacinamide). Nicotinamide is preferred according to the invention and is preferably contained in the agents according to the invention in quantities of from 0.05 to 1 wt. %, relative to the total agent.

Vitamin B₅ (pantothenic acid, panthenol and pantolactone). In the context of this group, panthenol and/or pantolactone are preferably used. Derivatives of panthenol which may be used according to the invention are in particular the esters and ethers of panthenol and cationically derivatized panthenols. Individual representatives are for example panthenol triacetate, the panthenol monoethyl ether and the monoacetate thereof and the panthenol derivatives disclosed in WO 92/13829.

Vitamin B₆ (pyridoxine as well as pyridoxamine and pyridoxal).

The stated compounds of the vitamin B group are contained in the agents according to the invention preferably in quantities of from 0.01 to 2 wt. %, relative to the total agent. Quantities of 0.03-1 wt. % are particularly preferred.

Vitamin C (ascorbic acid). Vitamin C is preferably used in the agents used according to the invention in quantities of from 0.01 to 3 wt. %, relative to the total agent. Use in the form of the palmitic acid ester, the glucosides or phosphates may be preferred. Use in combination with tocopherols may likewise be preferred.

Vitamin E (tocopherols, in particular α-tocopherol). Tocopherol and the derivatives thereof, which include in particular the esters such as the acetate, the nicotinate, the phosphate and the succinate, are preferably contained in the agents used according to the invention in quantities of from 0.01-1 wt. %, relative to the total agent.

Vitamin F. The term “vitamin F” is conventionally understood to mean essential fatty acids, in particular linoleic acid, linolenic acid and arachidonic acid.

Vitamin H. Vitamin H denotes the compound (3aS,4S,6aR)-2-oxohexahydrothienol[3,4-d]-imidazole-4-valeric acid, which is now known however by the common name biotin. Biotin is contained in the agents used according to the invention preferably in quantities of from 0.0001 to 1.0 wt. %, in particular in quantities of from 0.001 to 0.01 wt. %.

Preferably, the preparations used according to the invention contain vitamins, provitamins and vitamin precursors from groups A, E, F and H. It goes without saying that a plurality of vitamins and vitamin precursors may also be contained therein at the same time.

The total quantity of vitamins, provitamins, vitamin precursors and the derivatives thereof introduced into the agents according to the invention amounts, relative to the total weight of the agent, to 0.01 to 5 wt. %, preferably 0.02 to 4 wt. % and in particular 0.05 to 3 wt. %.

Protein hydrolysates for the purposes of the invention are understood to be protein hydrolysates and/or amino acids and the derivatives thereof (H). Protein hydrolysates are product mixtures which are obtained by acidically, basically or enzymatically catalyzed degradation of proteins. According to the invention, the term protein hydrolysates also covers total hydrolysates and individual amino acids and the derivatives thereof and mixtures of different amino acids. Furthermore, polymers built up from amino acids and amino acid derivatives are also covered according to the invention by the term protein hydrolysates. The latter include for example polyalanine, polyasparagine, polyserine etc. Further examples of compounds which may be used according to the invention are L-alanyl-L-proline, polyglycine, glycyl-Lglutamine or D/L-methionine-5-methylsulfonium chloride. It goes without saying that, according to the invention, β-amino acids and the derivatives thereof such as β-alanine, anthranilic acid or hippuric acid may also be used. The molecular weight of the protein hydrolysates which may be used according to the invention is between 75, the molecular weight of glycine, and 200000, the molecular weight preferably amounting to 75 to 50000 and very particularly preferably to 75 to 20000 daltons.

Protein hydrolysates of both plant and animal origin or marine or synthetic origin may be used according to the invention.

Animal protein hydrolysates are for example elastin, collagen, keratin, silk and milk protein hydrolysates which may also assume salt form. Such products are distributed for example under the tradenames Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

It is preferred according to the invention to use protein hydrolysates of plant origin, for example soy, almond, pea, potato and wheat protein hydrolysates. Such products are obtainable for example under the tradenames Gluadin® (Cognis), DiaMin® (Diamalt), Lexein® (Inolex), Hydrosoy® (Croda), Hydrolupin® (Croda), Hydrosesame®) (Croda), Hydrotritium® (Croda) and Crotein® (Croda).

Although the use of protein hydrolysates as such is preferred, amino acid mixtures obtained in other ways may also optionally be used in their stead. It is likewise possible to use derivatives of protein hydrolysates, for example in the form of the fatty acid condensation products thereof. Such products are distributed, for example, under the names Lamepon® (Cognis), Lexein® (Inolex), Crolastin® (Croda) or Crotein® (Croda).

The protein hydrolysates or the derivatives thereof are contained in the preparations used according to the invention preferably in quantities of from 0.1 to 10 wt. %, relative to the total agent. Quantities of 0.1 to 5 wt. % are particularly preferred.

According to the invention, preference is given surfactant-containing cleansing agents which contain extracts from green tea, oak bark, stinging nettle, witch hazel, hops, henna, chamomile, burdock root, horsetail, hawthorn, lime blossom, almond, aloe vera, pine-needle oil, horse chestnut, sandalwood, juniper, coconut, mango, apricot, lime, wheat, kiwi, melon, orange, grapefruit, sage, rosemary, birch, mallow, lady's smock, wild thyme, yarrow, thyme, melissa, restharrow, coltsfoot, vanilla, marsh mallow, meristem, ginseng and ginger root.

Extracts from green tea, almond, aloe vera, coconut, mango, apricot, lime, wheat, vanilla, kiwi and melon are particularly preferred according to the invention, with particular preference being given to extracts from aloe vera, vanilla, and melon.

Conventionally, these extracts are provided by extraction of the entire plant. However, in individual cases it may also be preferable to produce the extracts solely from the blossoms and/or leaves of the plant.

Extracting agents for producing the stated plant extracts include water, alcohols and mixtures thereof. Preferred alcohols are lower alcohols such as ethanol and isopropanol, but in particular polyhydric alcohols such as ethylene glycol and propylene glycol, both as sole extracting agent and in a mixture with water. Plant extracts based on water/propylene glycol in the ratio 1:10 to 10:1 have proven particularly suitable.

The plant extracts may be used according to the invention both in pure and in dilute form. Where used in dilute form, they conventionally contain approx. 2-80 wt. % of active substance and as solvent the extracting agent or extracting agent mixture used to isolate them.

The plant extracts are used in the cleansing agent according to the invention, relative to the weight thereof, in a quantity of from 0.01 to 5 wt. %, preferably 0.02 to 4 wt. % and in particular 0.05 to 3 wt. %.

Furthermore, in a preferred embodiment of the invention the action of the preparations may be increased by UV filters. UV filters to be used according to the invention are not subject to any general restrictions with regard to structure and physical properties. Rather, any UV filters usable in the field of cosmetics whose absorption maximum is in the UVA (315-400 nm), the UVB (280-315 nm) or the UVC (<280 nm) range are suitable. UV filters with an absorption maximum in the UVB range, in particular in the range from approx. 280 to approx. 300 nm, are particularly preferred.

The UV filters used according to the invention may for example be selected from substituted benzophenones, p-aminobenzoic acid esters, diphenylacrylic acid esters, cinnamic acid esters, salicylic acid esters, benzimidazoles and o-aminobenzoic acid esters.

Examples of UV filters usable according to the invention are 4-aminobenzoic acid, N,N,N-trimethyl-4-(2-oxoborn-3-ylidenemethyl)aniline methyl sulfate, 3,3,5-trimethylcyclohexyl salicylate (homosalate), 2-hydroxy-4-methoxybenzophenone (benzophenone-3; Uvinul®M 40, Uvasorb®MET, Neo Heliopan®BB, Eusolex®4360), 2-phenylbenzimidazole-5-sulfonic acid and the potassium, sodium and triethanolamine salts (phenylbenzimidazole sulfonic acid; Parsol®HS; Neo Heliopan®Hydro), 3,3′-(1,4-phenylenedimethylene)bis(7,7-dimethyl-2-oxobicyclo-[2.2.1]hept-1-ylmethanesulfonic acid) and the salts thereof, 1-(4-tert.-butylphenyl)-3-(4-methoxyphenyl)-propane-1,3-dione (butyl methoxydibenzoylmethane; Parsol®1789, Eusolex®9020), α-(2-oxoborn-3-ylidene)-toluene-4-sulfonic acid and the salts thereof, ethoxylated 4-aminobenzoic acid ethyl ester (PEG-25 PABA; Uvinul®P 25), 4-dimethylaminobenzoic acid 2-ethylhexyl ester (octyl dimethyl PABA; Uvasorb®DMO, Escalol®507, Eusolex®6007), salicylic acid 2-ethylhexyl ester (octyl salicylate; Escalol®587, Neo Heliopan®OS, Uvinul®018), 4-methoxycinnamic acid isopentyl ester (isoamyl p-methoxycinnamate; Neo Heliopan®E 1000), 4-methoxycinnamic acid 2-ethylhexyl ester (octyl methoxycinnamate; Parsol®MCX, Escalol®557, Neo Heliopan®AV), 2-hydroxy-4-methoxybenzophenone-5-sulfonic acid and the sodium salt thereof (benzophenone-4; Uvinul®MS 40; Uvasorb®S 5), 3-(4′-methylbenzylidene)-D,L-camphor (4-methylbenzylidene camphor; Parsol®5000, Eusolex®6300), 3-benzylidenecamphor (3-benzylidene camphor), 4-isopropylbenzyl salicylate, 2,4,6-trianilino(p-carbo-2′-ethylhexyl-1′-oxy)-1,3,5-triazine, 3-imidazol-4-yl acrylic acid and the ethyl esters thereof, polymers of N-{(2 and 4)-[2-oxoborn-3-ylidenemethyl]-benzyl}-acrylamide, 2,4-dihydroxybenzophenone (benzophenone-1; Uvasorb®20H, Uvinul®400), 1,1′-diphenylacrylonitrile acid 2-ethylhexyl ester (octocrylene; Eusolex®OCR, Neo Heliopan®Type 303, Uvinul®N 539 SG), o-aminobenzoic acid menthyl ester (menthyl anthranilate; Neo Heliopan®MA), 2,2′,4,4′-tetrahydroxybenzophenone (benzophenone-2; Uvinul®D-50), 2,2′-dihydroxy-4,4′-dimethoxybenzophenone (benzophenone-6), 2,2′-dihydroxy-4,4′-dimethoxybenzophenone-5-sodium sulfonate and 2-cyano-3,3-diphenylacryl acid 2′-ethylhexyl ester. Preference is given to 4-aminobenzoic acid, N,N,N-tri methyl-4-(2-oxoborn-3-ylidenemethyl)aniline methyl sulfate, 3,3,5-trimethyl cyclohexyl salicylate, 2-hydroxy-4-methoxybenzophenone, 2-phenylbenzimidazole-5-sulfonic acid and the potassium, sodium and triethanolamine salts thereof, 3,3′-(1,4-phenylenedimethylene)-bis(7,7-dimethyl-2-oxobicyclo-[2.2.1]hept-1-ylmethanesulfonic acid) and the salts thereof, 1-(4-tert.butylphenyl)-3-(4-methoxyphenyl)propane-1,3-dione, α-(2-oxoborn-3-ylidene)toluene-4-sulfonic acid and the salts thereof, ethoxylated 4-aminobenzoic acid ethyl ester, 4-dimethylaminobenzoic acid 2-ethylhexyl ester, salicylic acid 2-ethylhexyl ester, 4-methoxycinnamic acid isopentyl ester, 4-methoxycinnamic acid 2-ethylhexyl ester, 2-hydroxy-4-methoxybenzophenone 5-sulfonic acid and the sodium salt thereof, 3-(4′-methylbenzylidene)-D,L-camphor, 3-benzylidenecamphor, 4-isopropylbenzyl salicylate, 2,4,6-trianilino-(p-carbo-2′-ethylhexyl-1′-oxy)-1,3,5-triazine, 3-imidazol-4-ylacrylic acid and the ethyl esters thereof, polymers of N-{(2 and 4)-[2-oxoborn-3-ylidenemethyl]benzyl}-acrylamide. Very particular preference is given according to the invention to 2-hydroxy-4-methoxybenzophenone, 2-phenylbenzimidazole-5-sulfonic acid and the potassium, sodium and triethanolamine salts thereof, 1-(4-tert.-butylphenyl)-3-(4-methoxyphenyl)-propane-1,3-dione, 4-methoxycinnamic acid 2-ethylhexyl ester and 3-(4′-methyl benzylidene)-D, L-camphor.

Those UV filters are preferred whose molar extinction coefficient at the absorption maximum is above 15000, in particular above 20000.

It has additionally been found that, in the case of structurally similar UV filters, in many cases the water-insoluble compound displays for the purposes of the teaching according to the invention the greater action relative to those water-soluble compounds which differ therefrom by one or more additionally ionic groups. Those UV filters which are understood for the purposes of the invention to be water-insoluble are those which at 20° C. are only 1 wt. %, in particular no more than 0.1 wt. %, soluble in water. Furthermore, these compounds should be at least 0.1, in particular at least 1 wt. %, soluble in conventional cosmetic oil components at room temperature. The use of water-insoluble UV filters may therefore be preferred according to the invention.

According to a further embodiment of the invention, those UVfilters are preferred which comprise a cationic group, in particular a quaternary ammonium group.

These UV filters have the general structure U-Q.

The structural element U therein denotes a UV radiationabsorbing group. This group may be derived in principle from the known above-stated UV filters usable in the field of cosmetics, in which a group, generally a hydrogen atom, of the UV filter is replaced by a cationic group Q, in particular with a quaternary amino function.

Compounds from which the structural element U may be derived are for example

substituted benzophenones,

p-aminobenzoic acid esters,

diphenylacrylic acid esters,

cinnamic acid esters,

salicylic acid esters,

benzimidazoles and

o-aminobenzoic acid esters.

Structural elements U which are derived from cinnamic acid amide or from N,N-dimethylaminobenzoic acid amide are preferred according to the invention.

The structural elements U may in principle be selected such that the absorption maximum of the UV filters may lie both in the UVA (315-400 nm) and in the UVB (280-315 nm) or in the UVC (<280 nm) range. UV filters with an absorption maximum in the UVB range, in particular in the range from approx. 280 to approx. 300 nm, are particularly preferred.

Furthermore, the structural element U, also as a function of structural element Q, is preferably selected such that the molar extinction coefficient of the UV filter at the absorption maximum is above 15000, in particular above 20000.

The structural element Q preferably contains a quaternary ammonium group as the cationic group. This quaternary ammonium group may in principle be linked directly to the structural element U, such that the structural element U is one of the four substituents of the positively charged nitrogen atom. However, one of the four substituents on the positively charged nitrogen atom is preferably a group, in particular an alkylene group with 2 to 6 carbon atoms, which functions as a link between the structural element U and the positively charged nitrogen atom.

Advantageously, the group Q has the general structure —(CH₂)ₓ—N⁺R¹R²R³X⁻, in which x denotes an integer from 1 to 4, R¹ and R² mutually independently denote C₁₋₄ alkyl groups, R³ denotes a C₁₋₂₂ alkyl group or a benzyl group and X⁻ denotes a physiologically acceptable anion. In the context of this general structure, x preferably denotes the number 3, R¹ and R² in each case denote a methyl group and R³ denotes either a methyl group or a saturated or unsaturated, linear or branched, hydrocarbon chain with 8 to 22, in particular 10 to 18, carbon atoms.

Physiologically acceptable anions are for example inorganic anions such as halides, in particular chloride, bromide and fluoride, sulfate ions and phosphate ions and organic anions such as lactate, citrate, acetate, tartrate, methosulfate and tosylate.

Two preferred UV filters with cationic groups are the commercially obtainable compounds cinnamic acid amidopropyltrimethylammonium chloride (Incroquat®UV-283) and dodecyldimethylaminobenzamidopropyldimethylammonium tosylate (Escalol® HP 610).

It goes without saying that the teaching according to the invention also includes the use of a combination of two or more UV filters. In the context of this embodiment, the combination of at least one water-insoluble UV filter with at least one UV filter having a cationic group is preferred.

The UV filters (I) are conventionally contained in the agents used according to the invention in quantities of 0.1-5 wt. % relative to the total agent. Quantities of 0.4-2.5 wt. % are preferred.

According to a further preferred embodiment of the invention, the surfactant-containing cleansing agents moreover contain at least one further water-insoluble silicone, a water-insoluble silicone and/or an aminofunctionalized silicone.

Silicones suitable according to the invention bring about the most varied effects. For example, they simultaneously influence dry and wet combability, the feel of the dry and wet hair and its shine. The term silicones is understood by the person skilled in the art to mean a plurality of organo-silicon compounds of different structures.

Dimethiconols (S1) form the first group of silicones, which are particularly preferred according to the invention. The dimethiconols according to the invention may be both linear and branched and cyclic or cyclic and branched. Linear dimethiconols may be illustrated by the following structural formula (S1-I):

(SiOHR¹₂)—O—(SiR²₂—O—)ₓ—(SiOHR¹₂) (S1-I)

Branched dimethiconols may be illustrated by the following structural formula (S1-II):

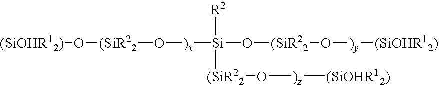

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; preferably R¹ and R² are an alkyl residue which contains 1 to approx. 6 carbon atoms, and most preferably R¹ and R² are methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. Preferably R¹ and R² are methyl, phenyl and C₂ to C₂₂ alkyl residues. The C₂ to C₂₂ alkyl residues are very particularly preferably lauryl, stearyl and behenyl residues. The numbers x, y and z are integers and run in each case mutually independently from 0 to 50000. The molar weights of the dimethicones are between 1000 D and 10000000 D. Viscosities are between 100 and 10000000 cPs measured at 25° C. with the assistance of a glass capillary viscometer using the Dow Corning Corporate Test Method CTM 0004 of 20 Jul. 1970. Preferred viscosities are between 1000 and 5000000 cPs, very particularly preferred viscosities are between 10000 and 3000000 cPs. The most preferred range is between 50000 and 2000000 cPs.

Of course, the teaching according to the invention also provides that the dimethiconols may already be present as an emulsion. In this case, the corresponding dimethiconol emulsion may be produced both after the production of the corresponding dimethiconols from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethiconol emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons, Inc. 1989. Reference is explicitly made to this standard work.

If the dimethiconols according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethiconols are used, it should be understood that the branching is greater in this case than the chance branching which arises due to impurities in the respective monomers. For the purposes of the present compound, branched dimethiconols should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethiconols with both a low and a high degree of branching may be very particularly preferred.

Exemplary commercial products are sold under the following marks: Botanisil NU-150M (Botanigenics), Dow Corning 1-1254 Fluid, Dow Corning 2-9023 Fluid, Dow Corning 2-9026 Fluid, Ultrapure Dimethiconol (Ultra Chemical), Unisil SF-R (Universal Preserve), X-21-5619 (Shin-Etsu Chemical Co.), Abil OSW 5 (Degussa Care Specialties), ACC DL-9430 Emulsion (Taylor Chemical Company), AEC Dimethiconol & Sodium Dodecylbenzenesulfonate (A & E Connock (Perfumery & Cosmetics) Ltd.), B C Dimethiconol Emulsion 95 (Basildon Chemical Company, Ltd.), Cosmetic Fluid 1401, Cosmetic Fluid 1403, Cosmetic Fluid 1501, Cosmetic Fluid 1401DC (all above-stated from Chemsil Silicones, Inc.), Dow Corning 1401 Fluid, Dow Corning 1403 Fluid, Dow Corning 1501 Fluid, Dow Corning 1784 HVF Emulsion, Dow Corning 9546 Silicone Elastomer Blend (all above-stated from Dow Corning Corporation), Dub Gel SI 1400 (Stearinerie Dubois Fils), HVM 4852 Emulsion (Crompton Corporation), Jeesilc 6056 (Jeen International Corporation), Lubrasil, Lubrasil DS (both from Guardian Laboratories), Nonychosine E, Nonychosine V (both from Exsymol), SanSurf Petrolatum-25, Satin Finish (both from Collaborative Laboratories, Inc.), Silatex-D30 (Cosmetic Ingredient Resources), Silsoft 148, Silsoft E-50, Silsoft E-623 (all above-stated from Crompton Corporation), SM555, SM2725, SM2765, SM2785 (all above-stated from GE Silicones), Taylor T-Sil CD-1, Taylor TME-4050E (all from Taylor Chemical Company), TH V 148 (Crompton Corporation), Tixogel CYD-1429 (Sud-Chemie Performance Additives), Wacker-Belsil CM 1000, Wacker-Belsil CM 3092, Wacker-Belsil CM 5040, Wacker-Belsil DM 3096, Wacker-Belsil DM 3112 VP, Wacker-Belsil DM 8005 VP, Wacker-Belsil DM 60081 VP (all above-stated from Wacker-Chemie GmbH).

If the dimethiconols (S1) are contained in the basic composition, these compositions contain 0.01 to 10 wt. %, preferably 0.1 to 8 wt. %, particularly preferably 0.25 to 7.5 wt. % and in particular 0.5 to 5 wt. % of dimethiconol relative to the composition.

“Dimethicones” (S2) are also particularly suitable according to the invention. These may be both linear and branched and cyclic or cyclic and branched. Linear dimethicones may be illustrated by the following structural formula (S2-I):

(SiR¹₃)—O—(SiR²₂—O—)ₓ—(SiR¹₃) (S2-I)

Branched dimethicones may be illustrated by the structural formula (S2-II):

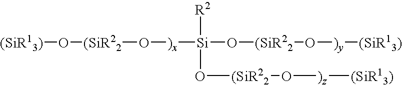

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; preferably R¹ and R² are an alkyl residue which contains 1 to approx. 6 carbon atoms, and most preferably R¹ and R² are methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. Preferably R¹ and R² are methyl, phenyl and C₂ to C₂₂ alkyl residues. The C₂ to C₂₂ alkyl residues are very particularly preferably lauryl, stearyl and behenyl residues. The numbers x, y and z are integers and run in each case mutually independently from 0 to 50000.

Particularly preferred according to the invention are linear polydialkylsiloxanes, polyalkylarylsiloxanes, polydiarylsiloxanes and/or dihydroxypolydimethylsiloxanes.

The molecular weights of the dimethicones suitable according to the invention are between 1000 D and 10000000 D.

The viscosities of the dimethicones suitable according to the invention are conventionally between 0.01 and 10000000 cPs measured at 25° C. with the assistance of a glass capillary viscometer using the Dow Corning Corporate Test Method CTM 0004 of 20 Jul. 1970. Preferred viscosities are between 0.1 and 5000000 cPs, very particularly preferred viscosities are between 0.1 and 3000000 cPs. The most preferred viscosity range of the dimethicones is between 0.6 and 600000 cPs.

Of course, the teaching according to the invention also provides that the dimethicones may already be present as an emulsion. In this case, the corresponding dimethicone emulsion may be produced both after the production of the corresponding dimethicones from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethicone emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons, Inc. 1989. Reference is explicitly made to this standard work.

If the dimethicones according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethicones are used, it should be understood that the branching is greater in this case than the chance branching which arises due to impurities in the respective monomers. For the purposes of the present compound, branched dimethicones should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethicones with both a low and a high degree of branching may be very particularly preferred.

If the dimethicones (S2) are contained in the basic composition, these compositions contain 0.01 to 10 wt. %, preferably 0.1 to 8 wt. %, particularly preferably 0.25 to 7.5 wt. % and in particular 0.5 to 5 wt. % of dimethicone relative to the composition.

Dimethicone copolyols (S3) form a further group of preferred silicones. Dimethiconols may be illustrated by the following structural formula:

(SiR¹₃)—O—(SiR²₂—O—)ₓ—(SiRPE—O—)_{y}—(SiR¹₃) (S3-I)

or by the following structural formula:

PE—(SiR¹₂)—O—(SiR²₂—O—)ₓ—(SiR¹₂)—PE (S3-II)

Branched dimethicone copolyols may be illustrated by the structural formula (S3-III):

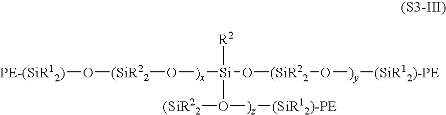

or by the structural formula (S3-IV):

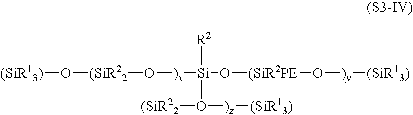

The residues R¹ and R² mutually independently denote in each case hydrogen, a methyl residue, a C₂ to C₃₀ linear, saturated or unsaturated hydrocarbon residue, a phenyl residue and/or an aryl residue. Non-limiting examples of the residues represented by R¹ and R² include alkyl residues, such as methyl, ethyl, propyl, isopropyl, butyl, isobutyl, pentyl, isopentyl, neopentyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like; R¹ and R² are preferably an alkyl residue containing 1 to approx. 6 carbon atoms, and R¹ and R² are most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—. Preferably R¹ and R² are methyl, phenyl and C₂ to C₂₂ alkyl residues. The C₂ to C₂₂ alkyl residues are very particularly preferably lauryl, stearyl and behenyl residues. PE denotes a polyoxyalkylene residue. Preferred polyoxyalkylene residues are derived from ethylene oxide, propylene oxide and glycerol. The numbers x, y and z are integers and run in each case mutually independently from 0 to 50000. The molar weights of the dimethicones are between 1000 D and 10000000 D. Viscosities are between 100 and 10000000 cPs measured at 25° C. with the assistance of a glass capillary viscometer using the Dow Corning Corporate Test Method CTM 0004 of 20 Jul. 1970. Preferred viscosities are between 1000 and 5000000 cPs, very particularly preferred viscosities are between 10000 and 3000000 cPs. The most preferred range is between 50000 and 2000000 cPs.

Of course, the teaching according to the invention also provides that the dimethicone copolymers may already be present as an emulsion. In this case, the corresponding dimethicone copolyol emulsion may be produced both after the production of the corresponding dimethicone copolyols from the latter and using the conventional methods of emulsification known to a person skilled in the art. To this end, any of cationic, anionic, nonionic or zwitterionic surfactants and emulsifiers may be used as auxiliary materials for producing the corresponding emulsions. It goes without saying that the dimethicone copolyol emulsions may also be produced directly by an emulsion polymerization method. Such methods are also well known to a person skilled in the art. In this respect, reference is made for example to the “Encyclopedia of Polymer Science and Engineering”, Volume 15, Second Edition, pages 204 to 308, John Wiley & Sons, Inc. 1989. Reference is explicitly made to this standard work.

If the dimethicone copolyols according to the invention are used in the form of an emulsion, the droplet size of the emulsified particles then amounts according to the invention to 0.01 μm to 10000 μm, preferably 0.01 to 100 μm, very particularly preferably 0.01 to 20 μm and most preferably 0.01 to 10 μm. Particle size is here determined using the light scattering method.

If branched dimethicone copolyols are used, it should be understood that the branching is greater in this case than the chance branching which arises due to impurities in the respective monomers. For the purposes of the present compound, branched dimethicone copolyols should therefore be taken to mean that the degree of branching is greater than 0.01%. Preferably, the degree of branching is greater than 0.1% and very particularly preferably greater than 0.5%. The degree of branching is determined in this case from the ratio of unbranched monomers, i.e. the quantity of monofunctional siloxane, to the branched monomers, i.e. the quantity of tri- and tetrafunctional siloxanes. According to the invention, dimethicone copolyols with both a low and a high degree of branching may be very particularly preferred.

If the dimethicone copolyols (S3) are contained in the basic composition, these compositions contain 0.01 to 10 wt. %, preferably 0.1 to 8 wt. %, particularly preferably 0.25 to 7.5 wt. % and in particular 0.5 to 5 wt. % of dimethicone copolyol relative to the composition.

Amino-functional silicones or amodimethicones (S4) are silicones which comprise at least one (optionally substituted) amino group.

Such silicones may, for example, be described by the formula (S4-I)

M(RₐQ_{b}SiO_{(4-a-b)/2)x}(R_{c}SiO_{(4-c)/2)y}M (S4-I)

wherein in the above formula R is a hydrocarbon or a hydrocarbon residue with 1 to approx. 6 carbon atoms, Q is a polar residue of the general formula —R¹HZ, in which R¹ is a divalent linking group, which is attached to hydrogen and the residue Z, composed of carbon and hydrogen atoms, carbon, hydrogen and oxygen atoms or carbon, hydrogen and nitrogen atoms, and Z is an organic, amino-functional residue, which contains at least one amino-functional group; “a” assumes values in the range from approx. 0 to approx. 2, “b” assumes values in the range from approx. 1 to approx. 3, “a”+“b” is less than or equal to 3, and “c” is a number in the range from approx. 1 to approx. 3, and x is a number in the range from 1 to approx. 2000, preferably from approx. 3 to approx. 50 and most preferably from approx. 3 to approx. 25, and y is a number in the range from approx. 20 to approx. 10000, preferably from approx. 125 to approx. 10000 and most preferably from approx. 150 to approx. 1000, and M is a suitable silicone end group, as known in the prior art, preferably trimethylsiloxy. Non-limiting examples of the residues represented by R include alkyl residues, such as methyl, ethyl, propyl, isopropyl, isopropyl, butyl, isobutyl, amyl, isoamyl, hexyl, isohexyl and the like; alkenyl residues, such as vinyl, halovinyl, alkylvinyl, allyl, haloallyl, alkylallyl; cycloalkyl residues, such as cyclobutyl, cyclopentyl, cyclohexyl and the like; phenyl residues, benzyl residues, halogenated hydrocarbon residues, such as 3-chloropropyl, 4-bromobutyl, 3,3,3-trifluoropropyl, chlorocyclohexyl, bromophenyl, chlorophenyl and the like and sulfur-containing residues, such as mercaptoethyl, mercaptopropyl, mercaptohexyl, mercaptophenyl and the like;
R is preferably an alkyl residue containing 1 to approx. 6 carbon atoms, and R is most preferably methyl. Examples of R¹ include methylene, ethylene, propylene, hexamethylene, decamethylene, —CH₂CH(CH₃)CH₂—, phenylene, naphthylene, —CH₂CH₂SCH₂CH₂—, —CH₂CH₂OCH₂—, —OCH₂CH₂—, —OCH₂CH₂CH₂—, —CH₂CH(CH₃)C(O)OCH₂—, —(CH₂)₃CC(O)OCH₂CH₂—, —C₆H₄C₆H₄—, —C₆H₄CH₂C₆H₄—; and —(CH₂)₃C(O)SCH₂CH₂—.

Z is an organic, amino-functional residue containing at least one functional amino group. A possible formula for Z is NH(CH₂)_{z}NH₂, in which z is 1 or more. Another possible formula for Z is —NH(CH₂)_{z}(CH₂)_{zz}NH, in which both z and zz are mutually independently an integer greater than or equal to 1, this structure comprising diamino ring structures, such as piperazinyl. Z is most preferably an —NHCH₂CH₂NH₂ residue. Another possible formula for Z is —N(CH₂)_{z}(CH₂)_{zz}NX₂ or —NX₂, in which each X of X₂ is independently selected from the group consisting of hydrogen and alkyl groups having 1 to 12 carbon atoms, and z is 0.

Q is most preferably a polar amino-functional residue of the formula —CH₂CH₂CH₂NHCH₂CH₂NH₂. In the formulae, “a” assumes values in the range from 0 to approx. 2, “b” assumes values in the range from approx. 2 to approx. 3, “a”+“b” is less than or equal to 3, and “c” is a number in the range from approx. 1 to approx. 3. The molar ratio of the RₐQ_{b} SiO_{(4-a-b)/2} units to the R_{c}SiO_{(4-c)/2} units is in the range from approx 1:2 to 1:65, preferably from approx. 1:5 to approx. 1:65 and most preferably from approx. 1:15 to approx. 1:20. If one or more silicones of the above formula are used, then the various variable substituents in the above formula may be different in the various silicone components which are present in the silicone mixture.

Preferred agents according to the invention are characterized in that they contain an amino-functional silicone of the formula (S4-II)

R′ₐG₃₋ₐ-Si(OSiG₂)ₙ-(OSiG_{b}R′_{2-b})ₘ—O—SiG₃₋ₐ-R′ₐ (S4-II),

in which:
G is —H, a phenyl group, —OH, —O—CH₃, —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃;
a denotes a number between 0 and 3, in particular 0;
b denotes a number between 0 and 1, in particular 1,
m and n are numbers, the sum of which (m+n) amounts to between 1 and 2000, preferably between 50 and 150, with n preferably assuming values from 0 to 1999 and in particular from 49 to 149 and m preferably assuming values from 1 to 2000, in particular from 1 to 10,
R′ is a monovalent residue selected from:
   —N(R″)—CH₂—CH₂—N(R″)₂
   —N(R″)₂
   —N⁺(R″)₃A⁻
   —N⁺H(R″)₂A⁻
   —N⁺H₂(R″)A⁻
   —N(R″)—CH₂—CH₂—N⁺R″H₂A⁻,
   wherein each R″ denotes identical or different residues from the group —H, -phenyl, -benzyl, C₁₋₂₀ alkyl residues, preferably —CH₃, —CH₂CH₃, —CH₂CH₂CH₃, —CH(CH₃)₂, —CH₂CH₂CH₂H₃, —CH₂CH(CH₃)₂, —CH(CH₃)CH₂CH₃, —C(CH₃)₃ and A represents an anion which is preferably selected from chloride, bromide, iodide or methosulfate.

Particularly preferred agents according to the invention are characterized in that they contain an amino-functional silicone of the formula (S4-III)

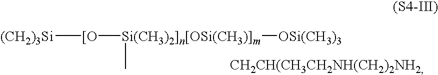

in which m and n are numbers, the sum of which (m+n) amounts to between 1 and 2000, preferably between 50 and 150, with n preferably assuming values from 0 to 1999 and in particular from 49 to 149 and m preferably assuming values from 1 to 2000, in particular from 1 to 10.

These silicones are denoted in accordance with the INCI Declaration as trimethylsilylamodimethicones.

Particularly preferred agents according to the invention are also characterized in that they contain an amino-functional silicone of the formula (S4-IV)

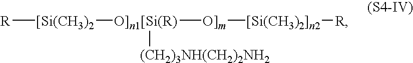

in which R denotes —OH, —O—CH₃— or a —CH₃ group and m, n1 and n2 are numbers the sum of which (m+n1+n2) amounts to between 1 and 2000, preferably between 50 and 150, wherein the sum (n1+n2) preferably assumes values from 0 to 1999 and in particular from 49 to 149 and m preferably assumes values from 1 to 2000, in particular from 1 to 10.

These silicones are denoted in accordance with the INCI Declaration as amodimethicones.

Irrespective of which amino-functional silicones are used, agents preferred according to the invention are those in which an amino-functional silicone has an amine value which is above 0.25 meq/g, preferably above 0.3 meq/g and in particular above 0.4 meq/g. The amine value here denotes milliequivalents of amine per gram of amino-functional silicone. It can be determined by titration and may also be stated in the unit mg of KOH/g.

If the amodimethicones (S4) are contained in the basic composition, these compositions contain 0.01 to 10 wt. %, preferably 0.1 to 8 wt. %, particularly preferably 0.25 to 7.5 wt. % and in particular 0.5 to 5 wt. % of amodimethicone relative to the composition.

If different silicones are used as a mixture, the mixing ratio is extensively variable. However, all the silicones used for the mixture are preferably used in a ratio of from 5:1 to 1:5 in the case of a binary mixture. A ratio of 3:1 to 1:3 is particularly preferable. Very particularly preferred mixtures contain all the silicones contained in the mixture very largely in a ratio of approx. 1:1, in each case relative to the quantities used in wt. %.

If the silicone mixture is contained in the basic composition, these compositions contain 0.01 to 10 wt. %, preferably 0.1 to 8 wt. %, particularly preferably 0.25 to 7.5 wt. % and in particular 0.5 to 5 wt. % of silicone mixture relative to the composition.

In a further preferred embodiment, the action of the active ingredient according to the invention may be enhanced by emulsifiers. Such emulsifiers are for example
addition products of 4 to 30 mol of ethylene oxide and/or 0 to 5 mol of propylene oxide onto linear fatty alcohols having 8 to 22 C atoms, onto fatty acids having 12 to 22 C atoms and onto alkylphenols having 8 to 15 C atoms in the alkyl group,
C₁₂-C₂₂ fatty acid mono- and diesters of addition products of 1 to 30 mol of ethylene oxide onto polyols having 3 to 6 carbon atoms, in particular onto glycerol,
ethylene oxide and polyglycerol addition products onto methyl glucoside/fatty acid esters, fatty acid alkanolamides and fatty acid glucamides,
C₈-C₂₂ alkyl mono- and oligoglycosides and the ethoxylated analogues thereof, with degrees of oligomerization of 1.1 to 5, in particular of 1.2 to 2.0, and glucose as the sugar component being preferred,
mixtures of alkyl (oligo)glucosides and fatty alcohols, for example the commercially available product Montanov®68,
addition products of 5 to 60 mol of ethylene oxide onto castor oil and hardened castor oil,
partial esters of polyols having 3 to 6 carbon atoms with saturated fatty acids having 8 to 22 C atoms,
sterols. Sterols are taken to be a group of steroids which bear a hydroxyl group on C atom 3 of the steroid skeleton and may be isolated both from animal tissue (zoosterols) and from vegetable fats (phytosterols). Examples of zoosterols are cholesterol and lanosterol. Examples of suitable phytosterols are ergosterol, stigmasterol and sitosterol. Sterols are also isolated from fungi and yeasts, these being known as mycosterols.
phospholipids. These are primarily taken to mean glucose phospholipids which are for example obtained as lecithins, or phosphatidylcholines for example from egg yolk or plant seeds (for example soy beans).
fatty acid esters of sugars and sugar alcohols, such as sorbitol,
polyglycerols and polyglycerol derivatives such as for example polyglycerol poly-12-hydroxystearate (commercial product Dehymuls® PGPH),
linear and branched fatty acids having 8 to 30 C atoms and the Na, K, ammonium, Ca, Mg and Zn salts thereof.

The agents according to the invention preferably contain the emulsifiers in quantities of 0.1-25 wt. %, in particular of 0.5-15 wt. %, relative to the total agent.

The compositions according to the invention may preferably contain at least one nonionogenic emulsifier with an HLB value of 8 to 18 in accordance with the definitions provided in Römpp-Lexikon Chemie (eds. J. Falbe, M. Regitz), 10th edition, Georg Thieme Verlag Stuttgart, New York, (1997), page 1764. Nonionogenic emulsifiers with an HLB value of 10-15 may be particularly preferred according to the invention.

It has additionally proven advantageous that further polymers can further support the action of the preparations according to the invention. In a preferred embodiment, the polymers are therefore added to the agents according to the invention, wherein both anionic, amphoteric and nonionic polymers have proven effective.

The anionic polymers which can support the action of the preparations according to the invention are anionic polymers which comprise carboxylate and/or sulfonate groups. Examples of anionic monomers of which such polymers may consist are acrylic acid, methacrylic acid, crotonic acid, maleic anhydride and 2-acrylamido-2-methylpropanesulfonic acid. In this case, the acidic groups may be present wholly or in part as a sodium, potassium, ammonium, mono- or triethanolammonium salt. 2-Acrylamido-2-methylpropanesulfonic acid and acrylic acid are preferred monomers.

Anionic polymers which have proven very particularly effective are those which contain as sole or co-monomer 2-acrylamido-2-methylpropanesulfonic acid, wherein the sulfonic acid group may be present wholly or in part as a sodium, potassium, ammonium, mono- or triethanolammonium salt.

The homopolymer of 2-acrylamido-2-methylpropanesulfonic acid is particular preferred, and is commercially available for example under the name Rheothik®11-80.

Within this embodiment it may be preferable to use copolymers of at least one anionic monomer and at least one non-ionogenic monomer. With regard to anionic monomers, reference is made to the above-listed substances. Preferred nonionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers and in particular polyacrylamide copolymers with monomers containing sulfonic acid groups. A particularly preferred anionic copolymer consists of 70 to 55 mol % acrylamide and 30 to 45 mol % 2-acrylamido-2-methylpropanesulfonic acid, the sulfonic acid group being present wholly or in part as a sodium, potassium, ammonium, mono- or triethanolammonium salt. This copolymer may also be present in crosslinked form, wherein polyolefinically unsaturated compounds such as tetraallyloxyethane, allyl sucrose, allyl pentaerythritol and methylene bisacrylamide are preferably used as the crosslinking agents. Such a polymer is contained in the commercial product Sepigel®305 from SEPPIC. Use of this compound, which contains in addition to the polymer component a hydrocarbon mixture (C₁₃-C₁₄ isoparaffin) and a non-ionogenic emulsifier (Laureth-7), has proven particularly advantageous for the purposes of the teaching according to the invention.

The sodium acryloyldimethyl taurate copolymers distributed under the name Simulgel®600 as a compound with isohexadecane and polysorbate-80 have also proven particularly effective according to the invention.

Anionic homopolymers which are likewise preferred are uncrosslinked and crosslinked polyacrylic acids. In this case, allyl ethers of pentaerythritol, of sucrose and of propylene may be preferred crosslinking agents. Such compounds are commercially available for example under the tradename Carbopol®.

Copolymers of maleic anhydride and methyl vinyl ether, in particular those comprising crosslinks, are also color-preserving polymers. A maleic acid-methyl vinyl ether copolymer crosslinked with 1,9-decadiene is commercially available under the name Stabileze® QM.

In addition, amphoteric polymers may be used as polymers for enhancing the action of the preparations according to the invention. The term amphoteric polymers includes not only those polymers which contain in each molecule both free amino groups and free —COOH— or SO₃H groups and are capable of forming internal salts, but also zwitterionic polymers, which contain in each molecule quaternary ammonium groups and —COO⁻ or —SO₃⁻ groups, and those polymers which contain —COOH— or SO₃H groups and quaternary ammonium groups.

One example of an amphoteric polymer usable according to the invention is the acrylic resin obtainable under the name Amphomer®, which is a copolymer of tert.-butylaminoethyl methacrylate, N-(1,1,3,3-tetramethylbutyl)acrylamide and two or more monomers from the group acrylic acid, methacrylic acid and the simple esters thereof.

Further amphoteric polymers usable according to the invention are the compounds stated in British published patent application 2 104 091, European published patent application 47 714, European published patent application 217 274, European published patent application 283 817 and German published patent application 28 17 369.

Amphoteric polymers which are preferably used are those polymers which are substantially composed of
(a) monomers with quaternary ammonium groups of the general formula (G3-I),

R¹—CH═CR²—CO-Z-(CₙH₂ₙ)—N⁽⁺⁾R³R⁴R⁵A⁽⁻⁾ (G3-I)

in which R¹ and R² mutually independently denote hydrogen or a methyl group and R³, R⁴ and R⁵ mutually independently denote alkyl groups having 1 to 4 carbon atoms, Z is an NH group or an oxygen atom, n is an integer from 2 to 5 and A⁽⁻⁾ is the anion of an organic or inorganic acid, and
(b) monomeric carboxylic acids of the general formula (G3-II),

R⁶—CH═CR⁷—COOH (G3-II)

in which R⁶ and R⁷ are mutually independently hydrogen or methyl groups.

These compounds may be used according to the invention both directly and in salt form, which is obtained by neutralization of the polymers, for example with an alkali metal hydroxide. With regard to the details of production of these polymers, explicit reference is made to the contents of German published patent application 39 29 973. Very particularly preferred polymers are those in which monomers of type (a) are used, in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (a). Acrylic acid is preferably used as monomer (b) for the stated polymers.

In a further embodiment, the agents according to the invention may contain nonionogenic polymers.

Suitable nonionogenic polymers are for example:
vinylpyrrolidone/vinyl ester copolymers, as are distributed for example under the tradename Luviskol® (BASF). Luviskol® VA 64 and Luviskol® VA 73, in each case vinylpyrrolidone/vinyl acetate copolymers, are likewise preferred nonionic polymers.
cellulose ethers, such as hydroxypropylcellulose, hydroxyethylcellulose and methylhydroxypropylcellulose, as are distributed for example under the tradename Culminal® and Benecel® (AQUALON).
shellac
polyvinylpyrrolidones, as are distributed for example under the tradename Luviskol® (BASF).
siloxanes. These siloxanes may be either water-soluble or water-insoluble. Both volatile and non-volatile siloxanes are suitable, wherein non-volatile siloxanes are taken to be those compounds whose boiling point at standard pressure is above 200° C. Preferred siloxanes are polydialkylsiloxanes, such as for example polydimethylsiloxane, polyalkylarylsiloxanes, such as for example polyphenylmethylsiloxane, ethoxylated polydialkylsiloxanes and polydialkylsiloxanes, which contain amine and/or hydroxy groups.
glycosidically substituted silicones according to EP 0612759 B1.

It is also possible according to the invention for the preparations used to contain a plurality of, in particular two different, identically charged polymers and/or in each case one ionic and one amphoteric and/or non-ionic polymer.

The polymers are contained in the agents used according to the invention preferably in quantities of from 0.05 to 10 wt. %, relative to the total agent. Quantities of 0.1 to 5, in particular of 0.1 to 3 wt. %, are particularly preferred.

Further active ingredients and auxiliary substances and additives are for example
thickeners such as agar agar, guar gum, alginates, xanthan gum, gum arabic, gum karaya, locust bean flour, linseed gums, dextrans, cellulose derivatives, for example methylcellulose, hydroxyalkylcellulose and carboxymethylcellulose, starch fractions and derivatives such as amylose, amylopectin and dextrins, clays such as for example bentonite or fully synthetic hydrocolloids, such as for example polyvinyl alcohol,
hair-conditioning compounds such as phospholipids, for example soy lecithin, egg lecithin and cephalins, as well as silicone oils,
perfume oils, dimethyl isosorbide and cyclodextrins,
solvents and solubilizing agents such as ethanol, isopropanol, ethylene glycol, propylene glycol, glycerol and diethylene glycol,
active ingredients which improve fiber structure, in particular mono-, di- and oligosaccharides such as for example glucose, galactose, fructose, fruit sugars and lactose,
defoamers such as silicones,
dyes for coloring the preparation,
further antidandruff active ingredients such as piroctone olamine, zinc omadine and climbazole,
active ingredients such as allantoin and bisabolol, cholesterol,
consistency providers, such as sugar esters, polyol esters or polyol alkyl ethers,
fats and waxes such as spermaceti, beeswax, montan wax and
paraffins,
fatty acid alkanolamides,
complexing agents such as EDTA, NTA, β-alaninediacetic acid and phosphonic acids,
swelling and penetrating substances such as primary, secondary and tertiary phosphates
opacifiers such as latex, styrene/PVP and styrene/acrylamide copolymers,
pearlescent agents such as ethylene glycol mono- and distearate and PEG-3 distearate,
pigments,
propellants such as propane/butane mixtures, N₂O, dimethyl ether, CO₂ and air,
antioxidants.

With regard to further optional components and the quantities of these components used, reference is explicitly made to the relevant handbooks known to a person skilled in the art, for example the monograph by K. H. Schrader, Grundlagen und Rezepturen der Kosmetika, 2nd edition, Hüthig Buch Verlag, Heidelberg, 1989.

The cleansing agents according to the invention are not subject to any limitations with regard to the form in which they are formulated and may be formulated as an emulsion, cream, solution, gel or mousse.

The present invention secondly provides a method of stabilizing insoluble antidandruff agents in surfactant systems, characterized in that hardened castor oil is introduced into the surfactant mixture containing the insoluble antidandruff agents.

In a particularly preferred embodiment of this method, the hardened castor oil is introduced in finely dispersed manner into the surfactant mixture by means of a hot process.

The present invention thirdly provides the use of hardened castor oil for stabilizing insoluble antidandruff agents in surfactant systems.

It is here particularly preferable to use the hardened castor oil which is introduced in finely dispersed manner into the surfactant mixture by means of a hot process.

The following Examples are intended to explain the invention in greater detail without restricting it to said Examples.

The quantities stated in the Examples relate, unless otherwise stated, to wt. %.

Other than where otherwise indicated, or where required to distinguish over the prior art, all numbers expressing quantities of ingredients herein are to be understood as modified in all instances by the term “about”. As used herein, the words “may” and “may be” are to be interpreted in an open-ended, non-restrictive manner. At minimum, “may” and “may be” are to be interpreted as definitively including, but not limited to, the composition, structure, or act recited.

As used herein, and in particular as used herein to define the elements of the claims that follow, the articles “a” and “an” are synonymous and used interchangeably with “at least one” or “one or more,” disclosing or encompassing both the singular and the plural, unless specifically defined herein otherwise. The conjunction “or” is used herein in both in the conjunctive and disjunctive sense, such that phrases or terms conjoined by “or” disclose or encompass each phrase or term alone as well as any combination so conjoined, unless specifically defined herein otherwise.

The description of a group or class of materials as suitable or preferred for a given purpose in connection with the invention implies that mixtures of any two or more of the members of the group or class are equally suitable or preferred. Description of constituents in chemical terms refers unless otherwise indicated, to the constituents at the time of addition to any combination specified in the description, and does not necessarily preclude chemical interactions among the constituents of a mixture once mixed. Steps in any method disclosed or claimed need not be performed in the order recited, except as otherwise specifically disclosed or claimed.

Changes in form and substitution of equivalents are contemplated as circumstances may suggest or render expedient. Although specific terms have been employed herein, such terms are intended in a descriptive sense and not for purposes of limitation.

### EXAMPLES

| Raw | | | | | | | | | | | |
|---|---|---|---|---|---|---|---|---|---|---|---|
| material | | Formulations (quantity) | | | | | | | | | |
| group | Raw material | A | B | C | D | E | F | G | H | I | J |
| 1 | Water | ad 100 | ad 100 | ad 100 | ad 100 | ad 100 | ad 100 | ad 100 | ad 100 | ad 100 | ad 100 |
| 1A | Sodium Laureth | 10.00 | 10.00 | | | 9.50 | 9.50 | 10.00 | 10.00 | 9.00 | 9.00 |
| | Sulfate | | | | | | | | | | |
| 1A | Sodium Lauryl | | | 2.50 | 2.50 | | | | | | |
| | Sulfate | | | | | | | | | | |
| 1A | Ammonium Laureth | | | 7.50 | 7.50 | | | | | | |
| | Sulfate | | | | | | | | | | |
| 1B | Hydrogenated | 0.50 | | 0.25 | | 0.30 | | 0.50 | | 0.30 | |
| | Castor Oil (Cutina | | | | | | | | | | |
| | HR) | | | | | | | | | | |
| 1C | Dimethicone | 0.60 | 0.60 | | | | | | | | |
| | 60,000 cSt. | | | | | | | | | | |
| 1C | Dimethicone | | | 1.30 | 1.30 | | | | | | |
| | 300,000 cSt. | | | | | | | | | | |
| 1D | Cetyl alcohol | | | | | | | 0.50 | 0.50 | 0.30 | 0.30 |
| 1D | Glycol Stearate | | | | | | | | | 0.50 | 0.50 |
| 1D | Glycol Distearate | 0.80 | 0.80 | | | 0.80 | 0.80 | | | 0.50 | 0.50 |
| 1D | Styrene/VP | | | 0.30 | 0.30 | | | | | | |
| | Copolymer | | | | | | | | | | |
| 1D | Titanium oxides | | | | | | | 0.10 | 0.10 | 0.10 | 0.10 |
| 1D | Mica | | | | | | | 0.10 | 0.10 | 0.10 | 0.10 |
| 2A | Water | | | | | | | 20.00 | 20.00 | 20.00 | 20.00 |
| 2B | Carbomer | | | | | | | 0.40 | 0.40 | | |
| 2B | Acrylates/C₁₀₋₃₀- | | | | | | | | | 0.40 | 0.40 |
| | Alkylacrylates | | | | | | | | | | |
| | Crosspolymer | | | | | | | | | | |
| 2B | Sodium Hydroxide | | | | | | | 0.20 | 0.20 | 0.20 | 0.20 |
| 3A | Water | 35.00 | 35.00 | 35.00 | 35.00 | 35.00 | 35.00 | 15.00 | 15.00 | 15.00 | 15.00 |
| 3B | Sodium Salicylate | 0.15 | 0.15 | | | 0.15 | 0.15 | | | 0.15 | 0.15 |
| 3B | Sodium Benzoate | 0.45 | 0.45 | | | 0.50 | 0.50 | | | 0.45 | 0.45 |
| 3B | Methylparabens/ | | | 0.30 | 0.30 | | | 0.30 | 0.30 | | |
| | Propylparabens/ | | | | | | | | | | |
| | Butylparabens | | | | | | | | | | |
| 3B | Methyl- | | | 0.01 | 0.01 | | | 0.01 | 0.01 | | |
| | isothiazolinone | | | | | | | | | | |
| 3C | Polyquaternium-10 | 0.15 | 0.15 | | | | | | | | |
| 3C | Polyquaternium-7 | | | 0.30 | 0.30 | | | | | 0.30 | 0.30 |
| 3C | Guar | | | | | 0.40 | 0.40 | | | | |
| | Hydroxypropyl | | | | | | | | | | |
| | Trimonium | | | | | | | | | | |
| | Chloride | | | | | | | | | | |
| 3D | Panthenol | 0.50 | 0.50 | | | 0.50 | 0.50 | 0.50 | 0.50 | 0.50 | 0.50 |
| 3D | Niacinamide | 0.30 | 0.30 | | | 0.30 | 0.30 | | | 0.30 | 0.30 |
| 3D | Hydrolyzed Wheat | | | 0.20 | 0.20 | | | 0.10 | 0.10 | | |
| | Protein | | | | | | | | | | |
| 3D | Pantolactone | | | 0.30 | 0.30 | | | | | | |
| 3D | Laurdimonium | | | | | | | 0.15 | 0.15 | | |
| | Hydroxypropyl | | | | | | | | | | |
| | Hydrolyzed Wheat | | | | | | | | | | |
| | Protein | | | | | | | | | | |
| 4A | Zinc Pyrithione 50% | 2.00 | 2.00 | 2.00 | 2.00 | 2.00 | 2.00 | 2.00 | 2.00 | 2.00 | 2.00 |
| 4A | Cocoamidopropyl | | | 3.50 | 3.50 | | | | | | |
| | Betaine | | | | | | | | | | |
| 4A | Disodium | 3.00 | 3.00 | | | | | 3.70 | 3.70 | | |
| | Cocoamphodiacetate | | | | | | | | | | |
| 4A | Coco-Betaine | | | | | 2.90 | 2.90 | | | | |
| 4A | Coco-Glucoside | | | | | | | | | 1.20 | 1.20 |
| 4A | Sodium | | | | | | | | | 3.00 | 3.00 |
| | Cocoamphoacetate | | | | | | | | | | |
| 4B | Laureth-2 | | | 0.30 | 0.30 | | | | | | |
| 4B | Propylene Glycol & | | | | | | | 0.10 | 0.10 | | |
| | PEG-55 Propylene | | | | | | | | | | |
| | Glycol Oleate | | | | | | | | | | |
| 4B | PEG-120 Methyl | 0.20 | 0.20 | | | | | | | | |
| | Glucose Dioleate | | | | | | | | | | |
| 4C | Perfume | 0.50 | 0.50 | 0.50 | 0.50 | 0.50 | 0.50 | 0.50 | 0.50 | 0.50 | 0.50 |
| 5A | Citric Acid | 0.1-0.5 | 0.1-0.5 | | | 0.1-0.5 | 0.1-0.5 | 0.1-0.5 | 0.1-0.5 | 0.1-0.5 | 0.1-0.5 |
| 5A | Lactic Acid | | | 0.1-0.5 | 0.1-0.5 | | | | | | |
| 6A | Sodium Chloride | 0.05-1.00 | 0.05-1.00 | 0.05-1.00 | 0.05-1.00 | 0.05-1.00 | 0.05-1.00 | 0.05-1.00 | 0.05-1.00 | 0.05-1.00 | 0.05-1.00 |
| | pH value | 5.3 | 5.3 | 6.0 | 6.0 | 5.0 | 5.0 | 6.5 | 6.5 | 5.0 | 5.0 |
| | Viscosity | 7000-9000 | 7000-9000 | 7000-9000 | 7000-9000 | 7000-9000 | 7000-9000 | 5000-7000 | 5000-7000 | 5000-7000 | 5000-7000 |
| | (Brookfield DV2; | | | | | | | | | | |
| | +20° C.; 20 rpm) | | | | | | | | | | |
| | Evaluation of | n.c. | s.p. | n.c. | s.p. | n.c. | s.p. | n.c. | s.s.p. | n.c. | s.s.p |
| | stability after | | | | | | | | | | |
| | 12 weeks' storage | | | | | | | | | | |
| | at 45° C. | | | | | | | | | | |

n.c.: no changes
s.p. (s.s.p.): separation phenomena (slight separation phenomena)

Production Instructions:
1: Introduce reaction mixture 1 and 1A and heat to 75-80° C.
   2: Melt reaction mixture 1B and add with stirring
   3: Add reaction mixture 1C with stirring
   4: Homogenize
   5: Add reaction mixture 1D with stirring
   6: Separately introduce reaction mixture 2A
   7: Leave reaction mixture 2A to swell with stirring in reaction mixture 2 and then neutralize to pH 7 with NaOH
   8: Add reaction mixture 2 (steps 6 to 7) to reaction mixture 1 (steps 1 to 5)
   9: Separately introduce reaction mixture 3A
   10: Dissolve reaction mixture 3B in reaction mixture 3A
   11: Swell reaction mixture 3C in reaction mixture 3
   12: Dissolve reaction mixture 3D in reaction mixture 3
   13: Add reaction mixture 3 (steps 9 to 12) to the batch (reaction mixture 1+reaction mixture 2) with stirring
   14: Add reaction mixture 4A to the batch with stirring
   15: Add reaction mixture 4B to the batch with stirring
   16: Add reaction mixture 4C to the batch with stirring
   17: The pH value of the batch is adjusted with reaction mixture 5A
   18: The viscosity of the batch is established with reaction mixture 6A

What is claimed:

1. A cleansing agent, comprising a surfactant, hardened castor oil, and an insoluble antidandruff agent stably dispersed in the cleansing agent.

2. The cleansing agent of claim 1, comprising at least one anionic surfactant and at least one of an amphoteric, zwitterionic, or nonionic surfactant.

3. The cleansing agent of claim 1, comprising 5% to 35% by weight of surfactant or surfactants.

4. The cleansing agent of claim 3, comprising 7% to 25% by weight of surfactant or surfactants.

5. The cleansing agent of claim 4, comprising 8% to 15% by weight of surfactant or surfactants.

6. The cleansing agent as claimed of claim 1, wherein the antidandruff agent comprises at least one of ketoconazole, climbazole, zinc pyrithione, salicylic acid, zinc carbonates, or piroctone olamine.

7. The cleansing agent of claim 1, comprising 0.05% to 2.0% by weight of the antidandruff agent or agents.

8. The cleansing agent of claim 7, comprising 0.25% to 1.5% by weight of the antidandruff agent or agents.

9. The cleansing agent of claim 8, comprising 0.5% to 1.0% by weight of the antidandruff agent or agents.

10. The cleansing agent of claims 7, wherein the antidandruff agent comprises zinc pyrithione.

11. The cleansing agent of claim 1, comprising 0.01% to 5% by weight of the hardened castor oil.

12. The cleansing agent of claim 11, comprising 0.05% to 3% by weight of the hardened castor oil.

13. The cleansing agent of claim 12, comprising 0.1% to 2% by weight of the hardened castor oil.

14. The cleansing agent of claim 1, comprising at least one cationic polymer.

15. The cleansing agent of claim 1, further comprising at least one water-insoluble oil component, vitamin, provitamin, protein hydrolysate, plant extract, UV filter, amino acid, water-insoluble silicone, water-soluble silicone, or amodimethicone.

16. The cleansing agent of claim 15, wherein the water-insoluble oil component comprises a mineral, vegetable, animal, or synthetic oil.

17. A method of stabilizing an insoluble antidandruff agent in a surfactant mixture, comprising adding hardened castor oil to the surfactant mixture containing the insoluble antidandruff agent.

18. The method of claim 17, comprising adding the hardened castor oil in finely dispersed form to the surfactant mixture in a hot process.
